(19) United States
(12) Patent Application Publication
(10) Pub. No.: US 2026/0240127 A1
(43) Pub. Date: Aug. 20, 2026

(54) GENETICALLY MODIFIED PIG PRODUCTION METHOD, ESTABLISHED SOMATIC CELL, AND RAW MATERIAL FOR PRODUCING GENETICALLY MODIFIED PIG

(71) Applicant: PORMEDTEC CO., LTD., Kawasaki-shi, Kanagawa (JP)

(72) Inventors: Hiroshi NAGASHIMA, Kawasaki-shi, Kanagawa (JP); Masahito WATANABE, Kawasaki-shi, Kanagawa (JP); Hitomi MATSUNARI, Kawasaki-shi, Kanagawa (JP)

(21) Appl. No.: 18/869,957

(22) PCT Filed: May 30, 2023
(86) PCT No.: PCT/JP2023/020165

§ 371 Date: Nov. 27, 2024

(30) Foreign Application Priority Data
May 31, 2022 (JP) 2022-088950

(51) Int. Cl. A01K 67/027; C12N 5/071; C12N 15/52; C12N 15/85; C12N 15/877; C12N 15/90

(52) U.S. Cl. CPC A01K 67/027; C12N 5/0602; C12N 15/52; C12N 15/85; C12N 15/877; C12N 15/90; A01K 2217/072; A01K 2217/206; A01K 2227/108; C12N 2510/00; C12N 2517/00; C12N 2800/107; C12N 2830/34

(57) ABSTRACT

The purpose of the present invention is to provide: a method for producing a genetically modified pig having, introduced selectively to a tissue or organ, a gene group for expressing a specific functional protein in the tissue or organ; somatic cells established therefrom; and a raw material for producing a genetically modified pig. Provided is a method for producing a genetically modified pig, the method comprising a step for introducing, into a safe harbor in a first gene-transfected cell, a gene cassette which includes a gene encoding a functional protein and includes a construct that enables the functional protein to be expressed by a recombinant enzyme. The first gene-introduced cell is a gene-introduced cell established from a gene-introduced pig that was gene-introduced by microfertilizing an egg with a liquid including a vector and pig sperm. The vector includes a promoter for causing expression in a specific tissue or organ, and a gene that encodes the recombinant enzyme that is downstream of and under the control of the promoter for causing expression in a specific tissue or organ.

### TECHNICAL FIELD

The present invention relates to a method for producing a genetically modified pig, somatic cells established therefrom, and a raw material for producing a genetically modified pig.

### BACKGROUND ART

There is a need to develop a genetically modified pig that can conditionally (facultatively) express a specific functional protein in a tissue- or organ-selective manner or even in a stage-selective manner in the tissue or organ.

### DISCLOSURE OF THE INVENTION

### Problems to be Solved by the Invention

Compared to, for example, rodents such as mice, breeding or crossing of pigs is more complicated. Therefore, the present inventors initially attempted to produce a genetically modified pig by introducing into a pig a vector including both a regulatory gene cassette for conditional gene expression and a functional gene cassette including a gene encoding a functional protein. However, the present inventors have found that the introduction of a vector including both a regulatory gene cassette for conditional gene expression and a functional gene cassette including a gene encoding a functional protein into a pig is lethal and kills the pig. As a result, the present inventors have found that it is difficult to produce a genetically modified pig into which a group of genes that allow the above-mentioned functional protein to be conditionally expressed in a specific tissue or organ has been introduced (see Comparative Example described below).

The present invention is made in view of the problem mentioned above in the prior art, and an object thereof is to provide a method for producing a genetically modified pig in which a group of genes for expressing a specific functional protein in a tissue- or organ-selective manner in the tissue or organ is introduced; somatic cells established therefrom; and a raw material for producing a genetically modified pig.

### Means for Solving the Problems

As a result of extensive studies to solve the above problem, the present inventors have found that a genetically modified pig in which a group of genes for conditionally expressing a specific functional protein in a tissue or organ is introduced can be produced by introducing the group of genes in two steps, namely, introducing a vector including a regulatory gene cassette for conditional gene expression and a functional gene cassette including a gene encoding a functional protein separately. In other words, the present inventors have found that a genetically modified pig in which a group of genes for conditionally expressing a specific functional protein in a tissue or organ is introduced can be produced by constructing a first transgenic cell through gene transfer by way of microfertilization in the presence of a vector including a regulatory gene cassette and then further introducing a functional gene cassette including a gene encoding a functional protein into the first transgenic cell. The present invention has been completed based on the above finding. That is, the present invention is as follows.

<1> A method for producing a genetically modified pig, the method including
introducing a gene cassette including a construct into a safe harbor region in a first transgenic cell, the construct including a gene encoding a functional protein, the functional protein being expressible by an action of a recombinant enzyme,
the first transgenic cell being a transgenic cell established from a transgenic pig having been transformed by way of microfertilization of an egg with a solution including a vector and pig sperm, and
the vector including a promoter expressed in a specific tissue or organ, and a gene encoding the recombinant enzyme, the gene being downstream of and under control of the promoter expressed in a specific tissue or organ.
   <2> The method according to <1>, in which the safe harbor region is a ROSA region.
   <3> The method described in <1>, in which the vector is a plasmid vector or an artificial chromosome vector.
   <4> The method according to <1>, including establishing a second transgenic pig cell by introducing the gene cassette into the safe harbor region.
   <5> The method according to <1>, including subjecting the established second transgenic pig cell to somatic cell cloning to obtain the genetically modified pig.
   <6> A somatic cell established from a genetically modified pig produced by the method according to any one of <1> to <6>.

<7> A raw material for producing a genetically modified pig, the raw material including
a first transgenic pig cell including a promoter expressed in a specific tissue or organ, and a gene encoding a recombinant enzyme, the gene being downstream of and under control of the promoter expressed in a specific tissue or organ,
the raw material being for use for introducing a gene cassette including a construct into a safe harbor region in the first transgenic cell, the construct including a gene encoding a functional protein, the functional protein being expressible by an action of a recombinant enzyme.
   <8> A use of a first transgenic pig cell including a promoter expressed in a specific tissue or organ, and a gene encoding a recombinant enzyme, the gene being downstream of and under control of the promoter expressed in a specific tissue or organ,
the use being for a raw material for use for introducing a gene cassette including a construct into a safe harbor region in the first transgenic cell, the construct including a gene encoding a functional protein, the functional protein being expressible by an action of the recombinant enzyme.

### Effects of the Invention

The present invention can provide a method for producing a genetically modified pig in which a group of genes for conditionally expressing a specific functional protein in a tissue- or organ-selective (preferably tissue- or organ-specific) manner in the tissue or organ is introduced; somatic cells established therefrom; and a raw material for producing a genetically modified pig.

### BRIEF DESCRIPTION OF THE DRAWINGS

FIG. 1 is a schematic diagram showing a CreERT2 expression vector constructed in Reference Example 1;

FIG. 2 shows electrophoretic diagrams showing the results of sex determination;

FIG. 3 shows electrophoretic diagrams showing the results of gene transfer determination;

FIG. 4 shows electrophoretic diagrams showing confirmed results of CreERT2 expression in a fetal metanephros;

FIG. 5 shows a graph showing results of copy number determination of transgenes in an individual (diploid genome);

FIG. 6 is a schematic diagram showing a gene cassette including a loxP-Venus gene constructed in Reference Example 2;

FIG. 7 is a schematic diagram showing a gene cassette including a loxP-DTA gene constructed in Reference Example 3;

FIG. 8 shows electrophoretic diagrams showing confirmed results of knock-in of the above-mentioned gene cassette into a ROSA region;

FIG. 9 shows pictures showing confirmed results of induction of cell death by tamoxifen in genetically modified pig cells in which a gene cassette including a loxP-DTA gene has been introduced into a porcine ROSA region;

FIG. 10 is a schematic diagram showing an all-in-one type vector 1 including both a mSix2 promoter and CreERT2, and a loxP-DTA gene in Comparative Example; and

FIG. 11 is a schematic diagram showing an all-in-one type vector 2 including both a mSix2 promoter and ERT2CreERT2, and a loxP-DTA gene in Comparative Example.

### PREFERRED MODE FOR CARRYING OUT THE INVENTION

Although embodiments of the present invention will be described hereafter in detail, the present invention is not limited to the embodiments below in any way and can be implemented with modifications as appropriate within the scope of the object of the present invention.

<<Method for producing genetically modified pig>>

A first aspect of the present invention is a method for producing a genetically modified pig including introducing a gene cassette including a construct into a safe harbor region in a first transgenic cell, the construct including a gene encoding a functional protein, the functional protein being expressible by an action of a recombinant enzyme. First transgenic cell: a transgenic cell established from a transgenic pig having been transformed by way of microfertilization of an egg with a solution including the below-mentioned vector and pig sperm. Vector: a vector including a promoter expressed in a specific tissue or organ, and a gene encoding a recombinant enzyme, the gene being downstream of and under control of the promoter expressed in a specific tissue or organ.

A genetically modified pig produced by the method according to the first aspect may also be referred to as a double transgenic pig (double genetically modified pig) since the pig has undergone a first gene transfer of the above-mentioned vector by way of the above-mentioned microfertilization and a gene transfer of the above-mentioned gene cassette to the above-mentioned safe harbor region (second gene transfer). In the present invention, the genetically modified pig is preferably a genetically modified pig that can conditionally express a specific functional protein in a tissue- or organ-selective manner in the tissue or organ.

The genetically modified pig produced by the method according to the first aspect includes a gene cassette including a construct in a safe harbor region, the construct including a gene encoding a functional protein, the functional protein being expressible by an action of a recombinant enzyme. The genetically modified pig produced by the method according to the first aspect also includes a promoter that is expressed in a specific tissue or organ, and a gene encoding the recombinant enzyme being downstream of and under control of the promoter. Thus, coexistence of the recombinant enzyme with the construct (preferably in a cell, more preferably in a nucleus) enables conditional (facultative (preferably recombinant enzyme expression-dependent)) expression of the functional protein. In other words, in the method according to the first aspect, a genetically modified pig that enables conditional (facultative (preferably recombinant enzyme expression-dependent)) expression of the functional protein can be produced by introducing the gene cassette into the safe harbor region in the first transgenic cell. As a result, the genetically modified pig produced by the method according to the first aspect can express the functional protein in a tissue- or organ-selective manner (preferably in a tissue- or organ-specific manner) in the tissue or organ.

For pigs, a large amount (high copy number) of regulatory gene cassettes is required for conditional gene expression, whereas only a small amount (low copy number) of functional gene cassettes each including a gene encoding a functional protein are sufficient. When gene transfer (transfection) sufficient for conditional expression by a regulatory gene cassette is achieved, a quantitative balance between the regulatory gene cassette and the functional gene cassette is not sufficiently appropriate for pigs due to a too-high copy number of the functional gene cassettes. The present inventors believe that this is a problem of the prior art that makes production of the above-mentioned genetically modified pig difficult. The present inventors also believe that occurrence of nonspecific expression (leaky expression) of the functional gene cassette is another problem of the prior art that makes production of the genetically modified pig difficult.

More specifically, for example, integration of a low copy number of an all-in-one type vector including both the regulatory gene cassette and the functional gene cassette (see Comparative Example below) results in insufficient expression of a gene expressing a recombinant enzyme in a tissue-specific manner and, in turn, insufficient expression of the gene encoding a functional protein. On the other hand, integration of a high copy number of an all-in-one type vector including both the regulatory gene cassette and the functional gene cassette results in lethally excessive expression of the gene encoding a functional protein due to leaky expression and, in turn, death of a pig. As a result, production efficiency (survival rate) of a genetically modified pig is affected.

For a genetically modified pig produced by the method according to the first aspect, a copy number of genes each encoding the functional protein is not particularly limited and, for example, may be one or more copies, preferably two or more copies, and further preferably four or more copies per individual pig (diploid genome). An upper limit of the copy number of genes each encoding the functional protein is not particularly limited and, for example, may be 200 copies or less, 100 copies or less, 550 copies or less, etc., per individual pig (diploid genome).

For a genetically modified pig produced by the method according to the first aspect, a copy number of genes each encoding the recombinant enzyme is not particularly limited and, for example, may be one or more copies, preferably two or more copies, more preferably four or more copies, further preferably seven or more copies, particularly preferably 15 or more, and especially preferably 30 or more copies per individual pig (diploid genome). An upper limit of the copy number of the recombinant enzymes is not particularly limited and, for example, may be 500 copies or less, 300 copies or less, 100 copies or less, etc., per individual pig (diploid genome).

For a genetically modified pig produced by the method according to the first aspect, a ratio of the copy number of genes each encoding the functional protein/the copy number of genes each encoding the recombinant enzyme may be 1/2 to 1/300, preferably 2/3 to 2/100, more preferably 3/5 to 3/50, and further preferably 4/6 to 4/20. This is from the viewpoint of improvement of production efficiency (survival rate) of a genetically modified pig.

In the first aspect, the above-mentioned construct is not particularly limited as long as it includes a gene encoding the functional protein and the functional protein can be expressed by an action of a recombinant enzyme and examples thereof include a nucleic acid construct such as a DNA construct, a cDNA construct, a RNA construct, etc. (preferably gene construct). In the first aspect, the above-mentioned construct preferably includes a specific sequence that is selectively (preferably specifically) recognized by the recombinant enzyme, and more preferably includes at least one pair of specific sequences that are selectively (preferably specifically) recognized by the recombinant enzyme. This allows the recombinant enzyme to selectively recognize the at least one pair of specific sequences (preferably at least one pair of specific sequences) to cause conditional (facultative (preferably recombinant enzyme expression-dependent)) recombination.

Examples of a combination of the recombinant enzyme with a specific sequence (preferably at least one pair of specific sequences) that the recombinant enzyme selectively recognizes include combinations (a) to (d) below, with the below-mentioned (a) combination of a Cre recombinase with at least one pair of Lox sequences being preferred:
(a) a combination of a Cre recombinase with at least one pair of Lox sequences;
(b) a combination of a recombinant enzyme FLP derived from budding yeast (Saccharomyces cerevisiae) with the below-mentioned FRT sequence (Broach, J. R., Guarascio, V. R., & Jayaram, M. (1982). Recombination within the yeast plasmid 2mu circle is site-specific. Cell, 29(1), 227-34.) FRT sequence: 5′-

(SEQ ID NO: 7)
GAAGTTCCTATTCTCTAGAAAGTATAGGAACTTC-3′;

(c) a combination of recombinant enzyme R derived from soy sauce yeast (Zygosaccharomyces rouxii) with a RS sequence (Araki, H., Jearnpipatkul, A., Tatsumi, H., Sakurai, T., Ushio, K., Muta, T., & Oshima, Y (1985). Molecular and functional organization of yeast plasmid pSR1. Journal of Molecular Biology, 182(2), 191-203.); and
(d) a combination of recombinant enzyme Gin derived from bacteriophage Mu with a gix sequence (Maeser, S., & Kahmann, R. (1991). The Gin recombinase of phage Mu can catalyse site-specific recombination in plant protoplasts. Molecular & General Genetics MGG, 230(1-2), 170-6.).

The specific sequence is preferably the Lox sequence, and among the Lox sequences, a loxP sequence of 34 bases in length derived from bacteriophage P₁ is a representative example and can be used to construct the so-called Cre-loxP system. An example of a pair of loxP sequences is shown below:

(SEQ ID NO: 1)
5′-ATAACTTCGTATAGCATACATTATACGAAGTTAT-3′

It can be seen from above that 13 bases at both ends, a Cre binding site, are symmetrical and 8 bases in a central region are asymmetrical. The loxP sequence is directional, and if a pair of Lox sequences are oriented in the same direction (same orientation), a sequence therebetween is excised as circular DNA by a Cre. The thus-excised circular DNA rarely returns to its original state, and such excision can be said to be substantially irreversible. On the other hand, if a pair of Lox sequences are oriented in opposite directions (opposite orientation), a sequence therebetween is inversed (orientation is reversed). Therefore, such inversion can be said to be reversible.

In the first aspect, at least one pair of Lox sequences is preferably 1 to 4 pairs of Lox sequences, more preferably 1 to 3 pairs of Lox sequences, and further preferably 1 or 2 pairs of Lox sequences. The at least one pair of Lox sequences include not only the loxP sequence described above as a representative example, but also a lox511 sequence, a lox2272 sequence, a loxFAS sequence, a loxRE sequence, a loxLE sequence, etc., and two or more pairs thereof may be used in combination.

An example of the lox511 sequence, the lox2272 sequence, the loxFAS sequence, the loxRE sequence, or the loxLE sequence is shown below. In the below-mentioned sequences, a lowercase letter denotes a base differing from that of the loxP sequence.

lox511:
(SEQ ID NO: 2)
5′-ATAACTTCGTATAGtATACATTATACGAAGTTAT-3′
lox2272:
(SEQ ID NO: 3)
5′-ATAACTTCGTATAGgATACtTTATACGAAGTTAT-3′
loxFAS:
(SEQ ID NO: 4)
5′-ATAACTTCGTATAtacctttcTATACGAAGTTAT-3′
lox RE:
(SEQ ID NO: 5)
5′-ATAACTTCGTATAGCATACATTATACGAAcggta-3′
lox LE:
(SEQ ID NO: 6)
5′-taccgTTCGTATAGCATACATTATACGAAGTTAT-3′

It has been known that a specific recombination occurs between loxP-loxP, lox511-lox511, or lox2272-lox2272, but not between loxP-lox511 or loxP-lox2272. This specific recombination property can be utilized to cause a flip-excision (FLEx) switch in which a target gene (gene encoding a functional protein) oriented in opposite orientation (inactive) is activated with an exon-flip by causing “irreversible” recombination by a Cre using two or more pairs of Lox sequences (oriented in opposite directions (opposite orientation) for each one pair of sequences) (Jikken Igaku, November 2014, Vol. 32, No. 18). This can overcome reversibility of inversion when a pair of Lox sequences are oriented in opposite directions (opposite orientations).

In the case of the FLEx switch, examples of a combination of two pairs of Lox sequences include a combination of one pair of loxP with one pair of lox2272, a combination of one pair of loxP with one pair of lox511, etc.

Apart from the FLEx switch, an example of an aspect using one pair of Lox sequences is described below. In the above-mentioned gene cassette, a target gene (gene encoding a functional protein) can be oriented in an active orientation and a (Floxed) STOP sequence sandwiched by one pair of Lox sequences oriented in the same direction (same orientation) can be arranged upstream of the target gene. Since the one pair of Lox sequences are oriented in the same direction (same orientation), the STOP sequence is substantially irreversibly excised by a Cre and thus the target gene (gene encoding a functional protein) can be expressed. The STOP sequence is not particularly limited as long as it is a transcription arrest sequence, and may be an SV40 poly A signal sequence and preferably a sequence in which three SV40 poly A signal cassettes are linked together.

From the viewpoint of prevention of nonspecific recombination (leakage), in addition to the STOP sequence, a Pause sequence may be further included as a (Floxed) sequence sandwiched by one pair of Lox sequences oriented in the same direction (same orientation), preferably downstream of the STOP sequence. The Pause sequence is not particularly limited, and examples thereof include a human α2 globin gene RNA polymerase II transcriptional pause signal sequence, etc.

In the method according to the first aspect, the recombinant enzyme is preferably a fusion protein with a nuclear translocation protein, that is, a gene encoding the recombinant enzyme is preferably directly or indirectly fused with a gene encoding a nuclear translocation protein. The nuclear translocation protein is not particularly limited as long as it can be translocated to a nucleus, and may be a protein with a nuclear translocation signal, in particular, preferably an estrogen receptor ER. It is more preferred that the recombinant enzyme be a Cre recombinase and the gene encoding a recombinant enzyme be a gene encoding a fusion protein including the Cre recombinase and an estrogen receptor ER. The Cre recombinase is known as an enzyme (enzyme derived from bacteriophage P₁) that recognizes a specific base sequence (e.g., a pair consisting of two Lox sequences) to cause DNA homologous recombination. The estrogen receptor ER is known to have a nuclear translocation signal. If the fusion protein is a fusion protein including a Cre recombinase and an estrogen receptor ER, a genetically modified pig in which a Cre recombinase portion in the fusion protein recognizes at least one pair of Lox sequences to cause conditional (facultative (preferably Cre expression-dependent)) recombination can be produced by introducing the gene cassette into a safe harbor region in a first transgenic cell obtained by way of microfertilization of an egg with a solution including the vector.

In the method according to the first aspect, it is more preferred that the estrogen receptor ER be ERT2 and the fusion protein be CreERT2 or ERT2CreERT2. A tamoxifen compound (tamoxifen, 4-hydroxytamoxifen (4-OHT), etc.) can bind to an ER in a cytoplasm and translocate to a nucleus. ERT2 is known to be a protein in which an ER is modified to bind only a tamoxifen compound and a nuclear translocation signal (with estrogen) is deleted. Both CreERT2 and ERT2CreERT2 are fusion proteins of the ERT2 and a Cre, which can translocate to a nucleus in the presence of a tamoxifen compound and can cause recombination by the action of the Cre.

(Feil R, Brocard J, Mascrez B, LeMeur M, Metzger D and Chambon P (1996): Ligand-activated site-specific recombination in mice. Proc Natl Acad Sci USA, 93: 10887-10890. 1996. Feil R, Wagner J, Metzger D and Chambon P (1997): Regulation of Cre recombinase activity by mutated estrogen receptor ligand-binding domains. Biochemical and Biophysical Research Communications, 237: 752-727. 1997.) In the first aspect, CreERT2 or ERT2CreERT2 can be used to cause conditional (e.g., stage-specific) recombination by a tamoxifen compound. In the first aspect, the estrogen receptor ER is preferably ERT2 and the fusion protein is preferably CreERT2 or ERT2CreERT2.

In a gene cassette in the first aspect, the above-mentioned functional protein is not particularly limited as long as it is a protein that exerts any function. Examples thereof include a protein that exerts any function such as a luminescence (e.g., fluorescence, UV light) function, a labeling (radiolabeling, any tag) function, a gene expression regulation function, a gene recognition function, a function lethal to a cell (e.g., acting on a cytoplasmic membrane to destroy it), etc. and more specifically, a fluorescent protein (e.g., Green Fluorescent Protein (GFP), a modified GFP such as Venus), a metabolism-related enzyme (e.g., thymidine kinase, a drug-metabolizing enzyme (CYP), etc.), a cell proliferation-related factor (e.g., a regulator of apoptosis BAX, a fibroblast growth factor FGF, etc.), a growth-related gene protein (e.g., a growth hormone receptor, an insulin-like growth factor receptor, etc.), a cell toxin, a suicide gene protein (e.g., iCaspase9), etc. The functional protein is preferably not a protein derived from a carcinogenic mutation, a carcinogenic protein, or a reporter protein for detecting their expression from the viewpoint of more reliable achievement of improved production efficiency (survival rate) of a genetically modified pig. A fluorescent protein is generally known to be cytotoxic. The cytotoxin is not particularly limited as long as it causes a lethal effect on a cell (e.g., acts on a cytoplasmic membrane and destroys it) and is preferably a polymeric toxin and more preferably a toxic protein. Specific examples of the cytotoxin include a diphtheria toxin, a θ toxin, streptolysin S, an α,δ toxin, leucocidin, etc., with a diphtheria toxin being preferred and a diphtheria toxin A being more preferred. A diphtheria toxin is a bacterial toxin produced by Corynebacterium diphtheriae and consists of two domains, A and B chains. The A chain serves as a toxin itself, while the B chain binds to a receptor on a cell surface and functions to take the A chain into a cell.

In a gene cassette in the first aspect, a gene encoding a cytotoxin may be a gene encoding the above-mentioned cytotoxin, preferably a gene encoding a diphtheria toxin, and more preferably a gene encoding a diphtheria toxin A (hereinafter simply referred to as “DTA”).

In order to allow the gene cassette to undergo transcription by an action of a promoter in the below-mentioned safe harbor region or a promoter upstream of the safe harbor region, a splicing acceptor sequence (splice acceptor sequence) including AG or the like may or may not be further included, preferably is further included, more preferably is further included upstream of a “construct including a gene encoding a functional protein, the functional protein being expressible by an action of a recombinant enzyme”, and further preferably is further included upstream of a “specific sequence and a gene encoding a functional protein” in a gene cassette in the first aspect.

In order to regulate expression of the functional protein, any promoter may or may not be further included upstream of a gene encoding the functional protein (preferably a specific sequence and a gene encoding the functional protein) in the gene cassette including a construct in the first aspect. The promoter may be a promoter that functions in any cell (preferably a wide variety of cells), preferably a CAG (cytomegalovirus immediate early enhancer-chicken R-actin hybrid) promotor, a CMV promotor, an RSV promoter, etc., and more preferably a CAG promoter.

A drug selection sequence for selection of a transgenic pig cell (gene knock-in pig cell) may or may not be further included in the gene cassette including a construct in the first aspect. If the drug selection sequence is further included, the sequence is preferably included downstream of a gene encoding a functional protein. The drug selection sequence is not particularly limited and examples thereof include a gene cassette composed of an SV40 promoter, puromycin N-acetyltransferase, and pA (poly A) of herpes simplex virus thymidine kinase (HSV-TK), etc.

A safe harbor region of the below-mentioned first transgenic pig cell into which a gene cassette according to the first aspect is introduced refers to a region in a genome into which a transgene can be safely inserted and may be, for example, a non-coding region in a genome into which a transgene can be safely inserted. The safe harbor region is preferably a porcine ROSA region or a porcine H11 region and more preferably a porcine ROSA region. The pig ROSA region is not particularly limited as long as it is a ROSA region and is preferably a ROSA region between exons 1 and 2 of a pig chromosome 13.

(First Transgenic Pig Cell in which Gene Cassette Including Construct is Introduced into Safe Harbor Region)

A method according to the first aspect includes introducing the above-mentioned gene cassette including the above-mentioned construct into a safe harbor region of a first transgenic cell. In the first aspect, the first transgenic cell is a transgenic cell established from a transgenic pig having been transformed by way of microfertilization of an egg with a solution including a vector and pig sperm,
the vector including a promoter expressed in a specific tissue or organ, and a gene encoding the recombinant enzyme, the gene being downstream of and under control of the promoter expressed in a specific tissue or organ. The present invention also relates to a raw material for producing a genetically modified pig, the raw material including a first transgenic pig cell including a promoter expressed in a specific tissue or organ, and a gene encoding a recombinant enzyme, the gene being downstream of and under control of the promoter expressed in a specific tissue or organ,
the raw material being for use for introducing a gene cassette including a construct into a safe harbor region in the first transgenic cell, the construct including a gene encoding a functional protein, the functional protein being expressible by an action of a recombinant enzyme. A second aspect of the invention is the raw material for producing a genetically modified pig.

In the first and second aspects, the first transgenic pig cell is preferably an established (e.g., cell-lined, cloned) transgenic pig cell from the viewpoint of stable culture and high cell uniformity. Moreover, when the cell is established, pre-establishment steps such as gene transfer by microfertilization of an egg with a solution including the vector and pig sperm can be omitted and a genetically modified pig capable of conditionally expressing the functional protein in a specific tissue or organ can be produced in one step (e.g., substantially one step) of introducing the above-mentioned gene cassette into a safe harbor region, which results in excellent industrial mass productivity of a genetically modified pig.

In the first and second aspects, the functional protein may be expressed by an action of the recombinant enzyme in cells all over the body of the pig (systemic expressibility) or in cells of a specific tissue or organ (localized expressibility) and preferably in at least a “specific tissue or organ” as defined by the above-mentioned vector. Compared to, for example, rodents such as mice, breeding or crossing of pigs is more complicated. The functional protein may preferably be expressed in cells all over the body of the pig (systemic expressibility) since a variety of genetically modified pigs in which a group of genes each for conditionally expressing the functional protein in a specific tissue or organ is introduced can be produced in one step (e.g., substantially one step) of introducing the gene cassette into safe harbor regions of a variety of first transgenic pig cells prepared by microfertilization in the coexistence of various vectors including various promotor expressed in various tissues or organs. If the gene cassette including the construct is introduced into a safe harbor region in a genome, it can be expressed in cells all over the body of the pig (systemically expressibility).

The first transgenic cell is not particularly limited as long as it is a transgenic pig cell established from a transgenic pig that has been transformed by way of microfertilization of an egg with a solution including the vector and pig sperm, and having a safe harbor region (preferably in a genome). Examples thereof include a fibroblast, a preadipocyte, an embryo-derived pluripotent cell, a somatic stem cell, an induced pluripotent stem cell, etc., with a fibroblast being preferred and a fetal fibroblast being more preferred.

### ((Transformation by Microfertilization))

The first transgenic cell is a transgenic pig cell established from a transgenic pig that has been transformed by way of microfertilization of an egg with a solution including the vector and pig sperm. Microfertilization of an egg (preferably ovum, more preferably mature ovum) with a solution including the vector and the pig sperm (also referred to as intracytoplasmic sperm injection; ICSI) enables transformation with the vector via microfertilization (first gene modification) (ICSI mediated gene transfer method). Microfertilization can be performed by injecting a solution including the vector and pig sperm into the egg (preferably mature ovum) via any method (e.g., injection, glass tube, capillary tube) to fertilize them. The above injection procedure may or may not be performed manually (e.g., manually under a microscope by a skilled person), or may be performed using a micromanipulator. The resulting fertilized egg may or may not be activated by sonication. The fertilized egg can then be implanted in the uterus of a recipient female (foster mother) to develop a transgenic pig (transgenic pig) individual.

A solution including the sperm and the vector is not particularly limited as long as the sperm and the vector can coexist, and examples thereof include any aqueous solution, any buffer solution, any culture solution, etc. at a pH of around 7.0 to 8.0, which may optionally include an inorganic salt, a buffering agent, a nutrient component, serum albumin, etc. A pig egg subjected to the microfertilization is preferably an ovum and more preferably a mature ovum.

A somatic cell may be obtained or taken by any method from a genetically modified pig produced by transformation by way of the microfertilization (first gene modification) and used as the first transgenic cell. In addition, the somatic cell may be used as a donor cell, which may be nuclear-transplanted into a recipient enucleated egg (oocyte) by a fusion method such as cell fusion, cultured, and then transplanted to impregnate a foster mother. Thus, a clone may be produced.

### ((Vector))

For the vector to be used for transformation by way of microfertilization in order to establish a first transgenic cell, examples of a specific organ or tissue include a viscus (e.g., a pancreas, a kidney, a ureter, a bladder, a liver, a heart, a stomach, or an intestine), a genitalium (e.g., an ovary or a testicle), a zygote, an embryo, a fetus, a bone marrow (e.g., hematopoiesis), a brain, eyes, a nose, a mouth, skin, a nerve, or a tissue therefrom, or an artificial tissue (a cell sheet such as a chondrocyte sheet, an organoid, or the like), with a viscus, a genitalium, a zygote, an embryo, or a fetus being preferred, a viscus or a genitalium being more preferred, and pancreas (e.g., a pancreatic islet), a kidney (e.g., a metanephros, particularly, an urinary organ including a metanephros, a ureter, or a bladder) being further preferred.

For the vector, a promoter expressed in the specific tissue or organ is not particularly limited as long as it is a promoter expressed in the specific tissue or organ and is preferably a promoter expressed in an early developmental stage such as a promoter expressed in a stem cell or a progenitor cell from the viewpoint of reduction of immunogenicity of the specific tissue or organ. Expression of a transgene all over the body of a transgenic pig would cause lethal damage all over the body and reduce production efficiency (survival rate) of a genetically modified pig. In contrast, use of a promoter expressed in the specific tissue or organ can limit expression of the transgene to the specific tissue or organ, which is not lethal and can improve production efficiency (survival rate) of the genetically modified pig. Examples of the promoter expressed in the specific tissue or organ include a promoter that regulates transcription of Six2 (hereinafter simply referred to as “Six2 promoter”), a promoter that regulates transcription of Sall1 (hereinafter simply referred to as “Sall1 promoter”), a Six2 promoter, or a FGF20 promoter, with a Six2 promotor or a Sall1 promoter being preferred. Both Six2 and Sall1 are known to be expressed in, for example, a kidney, particularly in a kidney progenitor cell, specifically in a metanephric mesenchymal region, and especially in a progenitor cell in a metanephric mesenchymal region. Six2 is known to be expressed in an undifferentiated metanephric mesenchymal region surrounding a tip of a ureteric bud, among others.

Examples of the vector in the first aspect include a plasmid vector, a viral vector, an artificial chromosome vector, etc., with a plasmid vector or an artificial chromosome vector being preferred. The plasmid vector or the viral vector includes any cloning vector. For example, a cloning vector such as the pCR4Blunt-TOPO vector (manufactured by Thermo Fisher Scientific) is commercially available and can be used in the present invention. Examples of an artificial chromosome vector include a bacterial artificial chromosome (BAC), a yeast artificial chromosome (YAC), a P1 phage-derived artificial chromosome (PAC), a porcine artificial chromosome, etc., with BAC, YAC, or PAC being preferred. The vector may be linear or cyclic, and a linear vector is preferred from the viewpoint of the below-mentioned microfertilization and amplification. A vector can be linearized by any method.

### ((Pig Sperm))

A sperm to be used for the microfertilization may be any pig sperm.

A method for introducing a gene cassette according to the first and second aspects into the safe harbor region (second gene modification method) may be a genome editing method. Preferably, the gene cassette and a genome editing tool are introduced into a cell (preferably into a nucleus) using any transgene transfer technique such as a lipofection method, an electroporation method, or a calcium phosphate method, etc., and then into the safe harbor region using a genome editing method.

Introduction using a genome editing method may be introduction using a genome editing tool including an artificial nuclease for genome editing such as CRISPR (Clustered Regularly Interspaced Short Palindromic Repeats)/Cas nuclease or introduction using a genome editing tool including an artificial restriction enzyme (artificial nuclease) such as a transcription activator-like effector nuclease (TALEN) and a zinc finger nuclease (ZFN).

A CRISPR/Cas nuclease includes a guide RNA and a Cas nuclease (preferably Cas9). At least a partial sequence in the safe harbor region to be targeted may be an oligonucleotide included in the safe harbor region.

A eukaryotic cell or eukaryote including the safe harbor region can be transfected with a guide RNA specific for at least a partial subsequence in the safe harbor region or a guide DNA encoding the guide RNA and a nucleic acid encoding a Cas nuclease or a composition containing a Cas nuclease to thereby introduce a gene cassette including the construct into the safe harbor region. A nucleic acid encoding a Cas nuclease or a Cas nuclease and a guide RNA or a DNA encoding a guide RNA can be transferred into a cell by various methods known in the art including, but not limited to, microinjection, electroporation, a DEAE-dextran treatment, lipofection, nanoparticle-mediated transfection, protein transduction domain-mediated transfection, virus-mediated gene delivery, and PEG-mediated transfection into a protoplast, etc. A nucleic acid encoding a Cas nuclease or a Cas nuclease and a guide RNA can be transferred into an organism by various methods known in the art for administering a gene or a protein such as injection. A nucleic acid encoding a Cas nuclease or a Cas protein can be transferred into a cell in a form of a complex with a guide RNA or separately. A Cas nuclease fused to a protein transduction domain such as Tat can also be efficiently delivered into a cell. Preferably, a eukaryotic cell or a eukaryote is simultaneously or sequentially transfected with a Cas9 nuclease and a guide RNA. Sequential transfection can be performed by a first transfection with a nucleic acid encoding a Cas nuclease, followed by a second transfection with a naked guide RNA. Preferably, the second transfection is performed after 3, 6, 12, 18, or 24 hours, but not limited thereto.

Expression of a guide RNA may be performed using a guide RNA expression unit. The guide RNA expression unit is preferably a CRISPR-Cas9-based transcription unit including a target sequence (at least a partial sequence in the safe harbor region) and a guide RNA, preferably has a promoter region for expression of the guide RNA (promoter of RNA polymerase III (e.g., a promoter selected from a U6 promoter and an H1 promoter)), a target sequence (at least a partial sequence in the safe harbor region), and a guide RNA, and is more preferably one in which a promoter, a sequence complementary to a target sequence (at least a partial sequence in the safe harbor region), and a guide RNA are seamlessly linked. A CRISPR/Cas nuclease can also use a Cas9 mutant that cuts only one strand of double-stranded DNA serving as a nickase to prevent off-targeting. A single-strand break Cas9 mutant is, for example, Cas9(D10A). For example, when a single-strand break Cas9 mutant is used in combination with a guide RNA having a target sequence complementary to one strand of a target DNA and a guide RNA having a target sequence complementary to the other strand very close to it, one strand is cleaved with a specificity of 20 bases and the other strand is further cleaved with a specificity of 20 bases. This means that the target DNA is cleaved with a specificity of 40 bases in total, which can significantly increase target specificity.

In the first aspect, establishment of a second transgenic pig cell by introducing the gene cassette into the safe harbor region is preferably included. A second transgenic pig cell (gene knock-in pig cell) obtained by introducing a gene cassette into the safe harbor region in the first and second aspects can be selected and established by any method including limiting dilution to less than one cell per well, genetic analysis, and drug resistance, etc.

References (a) to (c) below describe establishment of a genetically modified pig cell by genome editing and production of a genetically modified cloned pig by somatic cell cloning of the thus-established genetically modified pig cell.
(a) Masahito Watanabe, Kazuhiro Umeyama, Kazuaki Nakano, Hitomi Matsunari, Toru Fukuda, Kei Matsumoto, Susumu Tajiri, Shuichiro Yamanaka, Koki Hasegawa, Kazutoshi Okamoto, Ayuko Uchikura, Shuko Takayanagi, Masaki Nagaya, Takashi Yokoo, Hiromitsu Nakauchi and Hiroshi Nagashima (2022) Generation of heterozygous PKD1 mutant pigs exhibiting earlyonset renal cyst formation. Laboratory Investigation, 102:560-569; https://doi.org/10.1038/s41374-021-00717-z
(b) Hitomi Matsunari, Michiyo Honda, Masahito Watanabe, Satsuki Fukushima, Kouta Suzuki, Shigeru Miyagawa, Kazuaki Nakano, Kazuhiro Umeyama, Ayuko Uchikura, Kazutoshi Okamoto, Masaki Nagaya, Teruhiko Toyo-oka, Yoshiki Sawa, Hiroshi Nagashima (2020) Pigs with 6-sarcoglycan deficiency exhibit traits of genetic cardiomyopathy. Laboratory Investigation, 100:887-899; https://doi.org/10.1038/s41374-020-0406-7
(c) Masahito Watanabe, Kazuaki Nakano, Hitomi Matsunari, Taisuke Matsuda, Miki Maehara, Takahiro Kanai, Mirina Kobayashi, Yukina Matsumura, Rieko Sakai, Momoko Kuramoto, Gota Hayashida, Yoshinori Asano, Shuko Takayanagi, Yoshikazu Arai, Kazuhiro Umeyama, Masaki Nagaya, Yutaka Hanazono, Hiroshi Nagashima (2013) Generation of Interleukin-2 Receptor Gamma Gene Knockout Pigs from Somatic Cells Genetically Modified by Zinc Finger Nuclease-Encoding mRNA. PLOS ONE, Volume 8, Issue 10, e76478 Introduction of the gene cassette into the safe harbor region and establishment of the second genetically modified pig cell can be performed according to methods described in references (a) to (c).

### <<Genetically Modified Pig>>

The first aspect preferably further includes somatic cell cloning of the second transgenic pig cell established as mentioned above to obtain a genetically modified pig of interest. In the first aspect, somatic cell cloning (somatic cell cloning technology) to obtain a genetically modified cloned pig (gene knock-in cloned pig) from the second transgenic pig cell (gene knock-in cell) obtained as mentioned above (preferably, established) is not particularly limited. Somatic cell cloning technology refers to a technology for producing a clone in which a pig cell (preferably genetically-modified pig-cell) from which a clone is to be produced is cultured to obtain a donor cell, which is nuclear-transplanted into a recipient enucleated egg (oocyte) by a fusion method such as cell fusion, cultured, and then transplanted to impregnate a foster mother.

Specifically, in a first nuclear transplantation, the second genetically-modified pig-cell (gene knock-in pig cell) obtained (established) as mentioned above can be introduced into a mature, enucleated unfertilized egg. This introduction can be performed using a Sendai virus, cell fusion with electrical stimulation, or intracytoplasmic injection. A nucleus can then be formed by an activation treatment. This activation treatment includes strontium, ethanol, electrical stimulation, etc. The thus-formed nucleus can be introduced into a denucleated fertilized egg as a donor for a second nuclear transplantation. The above procedure may or may not be performed manually (e.g., manually under a microscope by a skilled person), or may be performed using a micromanipulator. The resulting genetically-modified pig cell-derived constructed egg is cultured and then developed into an implantable embryo, which is transplanted into a uterus of a foster mother to develop a genetically modified cloned pig (gene knock-in cloned pig) individual derived from a genetically modified pig cell (gene knock-in pig cell).

In addition, as described above, the above-mentioned references (a) to (c) describe production of a genetically modified cloned pig by somatic cell cloning of an established genetically modified pig cell, and a genetically modified cloned pig can be produced by somatic cell cloning of an established second genetically modified pig cell according to the method described in the references (a) to (c).

The recipient enucleated oocyte (oocyte) is preferably a recipient enucleated egg from any mammal such as pig, cattle, horse, sheep, goat, a primate (e.g., human, ape (chimpanzee, monkey, etc.)), a rodent (e.g., mouse, rat), more preferably a recipient enucleated egg from a mammal that is more similar to human in a characteristic such as physique than a rodent, further preferably a recipient enucleated egg from pig, sheep, goat, or a primate (e.g., human, ape), and even more preferably a recipient enucleated egg from pig. A foster mother to which the implantable embryo is transplanted is preferably any mammal such as pig, cattle, horse, sheep, goat, a primate (e.g., human, ape (chimpanzee, monkey, etc.)), a rodent (e.g., mouse, rat), more preferably a mammal that is more similar to human in a characteristic such as physique than a rodent, further preferably pig, sheep, goat, or a primate (e.g., human, ape), and even more preferably pig.

A “promoter expressed in a specific tissue or organ, and a gene encoding the recombinant enzyme, the gene being downstream of and under control of the promoter expressed in a specific tissue or organ” may be present (integrated) at any location in the genetically modified pig (preferably a cell of the genetically modified pig). For example, it may be present (integrated) in a cytoplasm, a genome, a mitochondrial genome, etc. of the genetically modified pig (preferably, a cell of the genetically modified pig) and preferably be present (integrated) in a cytoplasm or a genome. If present in a cytoplasm, they may be present in a form included in a plasmid. The “promoter expressed in a specific tissue or organ, and a gene encoding the recombinant enzyme, the being downstream of and under control of the promoter expressed in a specific tissue or organ” may or may not be present (integrated) in the safe harbor region including the gene cassette including the construct including a gene encoding a functional protein, but is preferably not present (integrated) in the safe harbor region from the viewpoint of a balance of expression levels between a regulatory gene cassette and a functional gene cassette.

A genetically modified pig (double genetically modified pig) produced by a method according to the first aspect may be any of a fetus, a juvenile, or an adult, but is preferably a fetus from the viewpoint of low immunogenicity.

As mentioned above, a genetically modified pig produced by a method according to the first aspect can conditionally express a specific functional protein in a tissue- or organ-selective manner (preferably in a tissue- or organ-specific manner) in the tissue or organ. When the functional protein is a cytotoxin, a genetically modified pig produced by a method according to the first aspect can conditionally kill at least a portion of cells in a tissue or organ (preferably, cells inside the tissue or organ excluding cells on a surface or a surface layer of the tissue or organ). This allows at least a portion of cells inside only a desired tissue or organ (preferably, cells inside the tissue or organ excluding cells on a surface or a surface layer of the tissue or organ) to be conditionally killed.

As mentioned above, a genetically modified pig produced by a method according to the first aspect can also express a specific functional protein in a conditional and stage-specific manner. When the functional protein is a cytotoxin, a genetically modified pig produced by a method according to the first aspect can also kill at least a portion of cells in a tissue or organ (preferably, cells inside the tissue or organ excluding cells on a surface or a surface layer of the tissue or organ) in a conditional and stage-specific manner.

A genetically modified pig (double genetically modified pig) produced by a method according to the first aspect may or may not be a material for somatic cell cloning. Specifically, a somatic cell may be obtained or taken by any method from a genetically modified pig (double genetically modified pig) produced by a method according to the first aspect and used as a donor cell, which is nuclear-transplanted into a recipient enucleated egg (oocyte) by a fusion method such as cell fusion, cultured, and then transplanted to impregnate a foster mother. Thus, a clone may be produced. The present invention is also a somatic cell established (e.g., cloned) from a genetically modified pig (double genetically modified pig) produced by a method according to the first aspect. The above-mentioned somatic cell, when established, is stable in culture and highly uniform. As a result, a clone of a genetically modified pig (double genetically modified pig) produced by a method according to the first aspect is produced with excellent industrial mass productivity. A genetically modified pig (double genetically modified pig) produced by a method according to the first aspect may include the clone. A tissue or organ can be industrially produced from a genetically modified pig (double genetically modified pig) produced by a method according to the first aspect by using the genetically modified pig (double genetically modified pig) produced by a method according to the first aspect as a material for such somatic cell cloning.

Examples of the organ or tissue include a viscus (e.g., a pancreas, a kidney, a ureter, a bladder, a liver, a heart, a stomach, or an intestine), a genitalium (e.g., an ovary or a testicle), a zygote, an embryo, a fetus, a bone marrow (e.g., hematopoiesis), a brain, eyes, a nose, a mouth, skin, a nerve, or a tissue therefrom, or an artificial tissue (a cell sheet such as a chondrocyte sheet, an organoid, or the like), with a viscus, a genitalium, a zygote, an embryo, or a fetus being preferred, a viscus or a genitalium being more preferred, and pancreas (e.g., a pancreatic islet), a kidney (e.g., a metanephros, particularly, an urinary organ including a metanephros, a ureter, or a bladder) being further preferred.

When the above-mentioned functional protein is a cytotoxin, at least a portion of the cells (preferably, cells inside the tissue or organ excluding cells on a surface or a surface layer of the tissue or organ) can be conditionally killed and replaced with an exogenous substance. Here, the exogenous substance means a substance derived from other than the above-mentioned tissue or organ, such as a human- or animal-derived substance, a drug (e.g., medicine), or the like. The substances include, for example, a cell, a growth factor, a hormone, a cytokine, etc., with a cell being preferred. The cell may or may not be an established cell (e.g., a recombinant cell, an ES cell, an iPS cell).

### EXAMPLES

Hereinafter, the present invention will be more specifically described with reference to Examples of the present invention, but the present invention is not limited to thereto and can be variously applied without departing from the technical idea of the present invention.

### Reference Example 1

### (Construction of Tissue- or Organ-Specific CreERT2 Expression Vector)

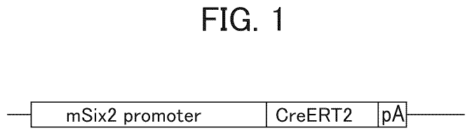

A gene cassette composed of a mSix2 promoter, which is about 7 kilobases upstream of a start codon peripheral region of a mouse Six2 gene, and CreERT2 and pA linked to a 3′ end of the mSix2 promoter was introduced into the pCR4Blunt-TOPO vector (manufactured by Thermo Fisher Scientific) to construct a tissue- or organ-specific CreERT2 expression vector. FIG. 1 is a schematic diagram showing the CreERT2 expression vector constructed as described above. In FIG. 1, pA represents a 3′-UTR of a rabbit β-globin gene. The resulting tissue- or organ-specific CreERT2 expression vector was linearized and then used in the below-mentioned microfertilization method (ICSI-mediated gene transfer method).

### (Construction of Transgenic Pig Using ICSI-Mediated Tissue- or Organ-Specific CreERT2 Expression Vector)

Sperms to be used in the microfertilization method were subjected to an ultrasonic treatment to separate a head from a tail. The ultrasonic treatment was performed at an intensity that caused separation of the head from the tail in about 70% of the sperms. After the tissue- or organ-specific CreERT2 expression vector constructed as mentioned above and the heads of pig sperms were co-cultured in a culture solution, one sperm head was sucked into an injection pipette, which was injected into an ovum using one or more piezoelectric pulses. Once confirming that the injection pipette had reached ooplasm, the sperm head was quickly injected into the ovum. The sperm was injected in an egg manipulation solution.

Preimplantation culture: After the ICSI, the ovum into which the sperm had been injected was transferred into a PZM5 culture drop prepared on a plastic culture dish (manufactured by AGC TECHNO GLASS CO., LTD.). A normal one-cell stage embryo and a normally-divided embryo were observed at 20 and 44 hours of culture. Alternatively, at 116 and 140 hours, a blastocyst stage embryo was observed.

Embryo transplantation: The embryo cultured in microdroplets of a PZM culture solution for the above-mentioned periods was transplanted into an oviduct or a uterus of a recipient pig. The normal one-cell stage embryo and the normally-divided embryo were transplanted to an oviduct within 1 day after ovulation, and the blastocyst was transplanted to a uterus 4 days after ovulation.

### (Sex Determination Test)

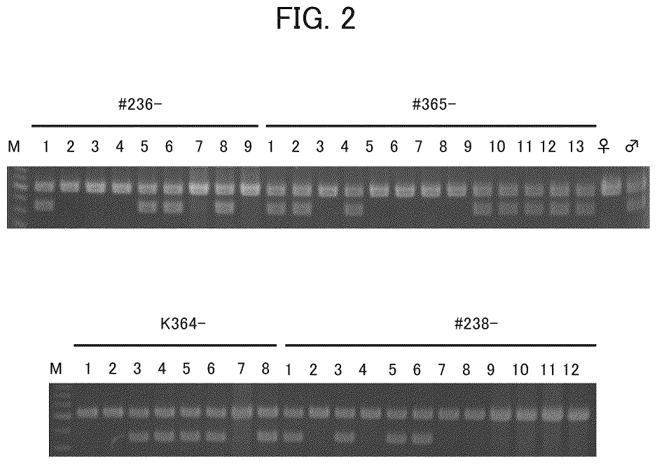

A total of 42 fetuses were obtained from four recipient pigs (#236, #365, K364, and #238). After a tail was taken from each of the fetuses, genomic DNA was extracted with a DNA extraction kit (DNeasy Blood and Tissue Kit, manufactured by QIAGEN) and subjected to sex determination by PCR using primers for amelogenin (AMEL X and AMEL Y). Electrophoresis diagrams of the results of sex determination for the 42 fetuses are shown in FIG. 2. As is clear from FIG. 2, #236-1, 5, 6, and 8, #365-1, 2, 4, 9, 10, 11, 12, and 13, K364-3, 4, 5, 6, and 8, and #238-1, 3, 5, and 6 were males and #236-2, 3, 4, 7, and 9, #365-3, 5, 6, 7, and 8, K364-1, 2, and 7, and #238-2, 4, and 7 to 12 were females.

### (Gene Transfer Determination Test)

The (above-mentioned) genomic DNA of each of the 42 fetuses was used to perform PCR using primers for a region from the mSix2 promoter to the CreERT2 on a transgene. The resulting PCR product was subjected to a gene transfer determination test by electrophoresis on 1% agarose gel (Tris-acetate-EDTA). The results are shown in Table 3.

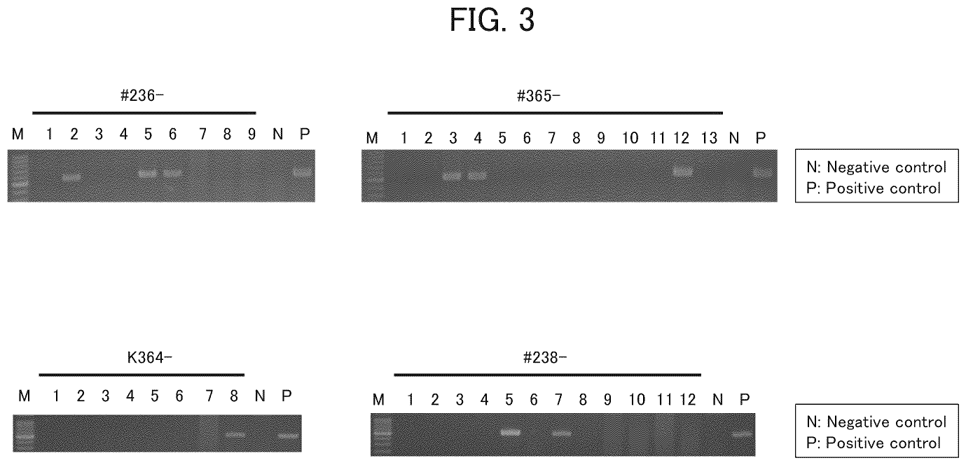

FIG. 3 shows electrophoretic diagrams showing the results of gene transfer determination. In FIG. 3, M denotes a DNA molecular weight marker, N denotes a negative control, i.e., wild-type porcine genomic DNA, and P denotes a positive control, i.e., CreERT2-expressing gene vector. As is clear from FIG. 3, a total of 9 tissue- or organ-specific CreERT2-expressing transgenic pig fetuses were obtained out of the 42 fetuses, and a production efficiency of the transgenic pig was 21% (#236-2, 5, and 6, #365-3, 4, and 12, K364-8, and #238-5 and 7). However, #365-12 and K364-8 were regressed fetuses.

### (CreERT2 Expression in Fetal Metanephros Confirmation Test)

One metanephros was taken from each of #236-2, 5, and 6 and #365-3, 4, and 12 out of the tissue- or organ-specific CreERT2-expressing transgenic pig fetuses and homogenized in a tissue solution attached to the RNeasy Plus Mini Kit (manufactured by QIAGEN) using a biomasher (manufactured by Nippi, Incorporated), from which a total RNA was collected according to the product protocol. A reverse transcription reaction was performed with the SuperScript III Reverse Transcriptase (manufactured by Thermo Fisher Scientific) to thereby obtain cDNA. An amplification reaction was performed using CreERT2 and PigSix2 primers in the Veriti thermal cycler (manufactured by Thermo Fisher Scientific) and a confirmation test of CreERT2 expression in a fetal metanephros was performed by RT-PCR (nested PCR). The resulting PCR product was subjected to a confirmation test of CreERT2 expression in a fetal metanephros by electrophoresis on 1% agarose gel (Tris-acetate-EDTA). The results are shown in Table 4.

CreERT2 primer sequence
1^{st}:
(SEQ ID NO: 9)
5′-CATGTCCATGCTGCCCACCTTCG-3′/
(SEQ ID NO: 10)
5′-GTATATCCTGGCAGCGATCGC-3′
2^{nd}:
(SEQ ID NO: 11)
5′-TCGCAAGAACCTGATGGACA-3′/
(SEQ ID NO: 12)
5′-CGCCGCATAACCAGTGAAAC-3′
PigSix2 primer sequence
1^{st}:
(SEQ ID NO: 13)
5′-AGTCGCAGCGTGCTGCGCGAG-3′/
(SEQ ID NO: 14)
5′-CTAGGAGCCCAGGTCCACGA-3′
2^{nd}:
(SEQ ID NO: 15)
5′-ACCACGCAGGTCAGCAACTGG-3′/
(SEQ ID NO: 16)
5′-TCCGAGCTGCCTAGCACCGAC-3′

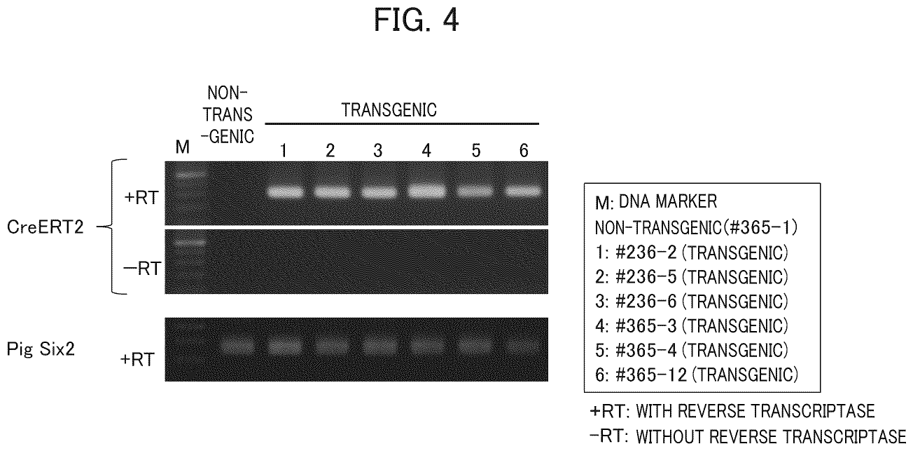

FIG. 4 shows electrophoretic diagrams showing confirmed results of CreERT2 expression in a fetal metanephros. In FIG. 4, +RT denotes data with a reverse transcriptase for sample amplification. As is clear from FIG. 4, CreERT2 gene expression under the mouse Six2 promoter was confirmed in a metanephros of all fetuses (#236-2, 5, and 6 and #365-3, 4, and 12) of the tissue- or organ-specific CreERT2-expressing transgenic pig fetuses.

### (Copy Number Determination of Transgene)

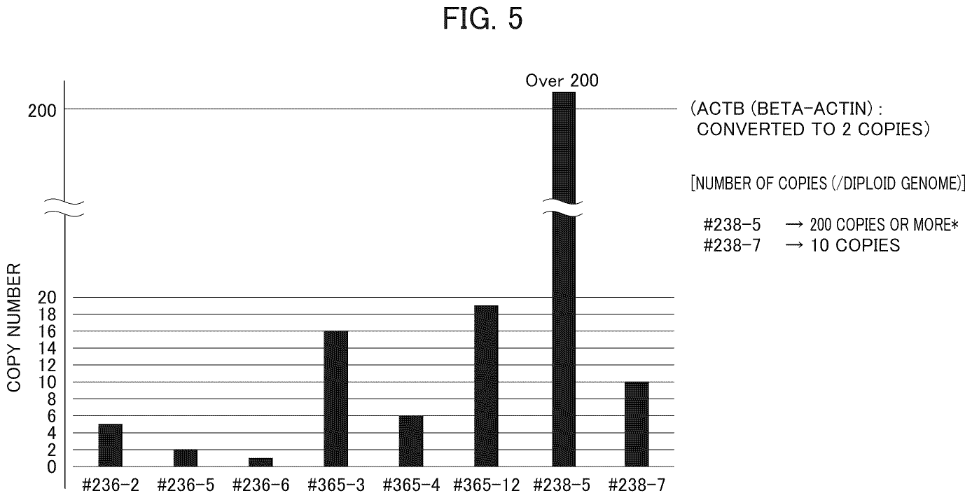

A genomic DNA was extracted from a tail tissue of each of #236-2, 5, and 6, #365-3, 4, and 12, and #238-5 and 7 of the tissue- or organ-specific CreERT2-expressing transgenic pig fetuses. A copy number of transgenes (introduced genes) in the genome was determined by digital PCR. The results are shown in Table 5. FIG. 5 shows a graph showing the results of copy number determination of transgenes in a diploid genome. In FIG. 5, β-actin serves as an internal standard (2 copies).

As is clear from the results shown in FIG. 5, in the diploid genome, a copy number of transgenes in #236-2 was 5 copies, a copy number of transgenes in #236-5 was 2 copies, a copy number of transgenes in #236-6 was 1 copy, a copy number of transgenes in #365-3 was 16 copies, a copy number of transgenes in #365-4 was 6 copies, and a copy number of transgenes in #365-12 was 19 copies, a copy number of transgenes in #238-5 was over 200 copies, and a copy number of transgenes in #238-7 was 10 copies. As described above, transgenic porcine fetuses heterozygously transformed with one, two, or more copies, or even 200 or more copies of the tissue- or organ-specific CreERT2 gene per individual (diploid) were obtained.

### Reference Example 2

(Construction of Gene Cassette Including loxP-Venus Gene)

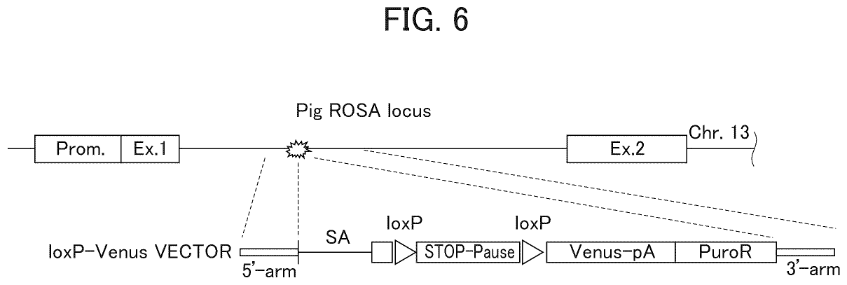

A knock-in vector composed of a splicing acceptor sequence, followed by a loxP-STOP-Pause-loxP sequence, and Venus-pA and PuroR (puromycin resistance gene cassette) for drug selection downstream thereof was constructed. Sequences of a ROSA region (about 700 base pairs each) as homology arms for knock-in are linked to both ends (5′ and 3′ sides) of the vector. FIG. 6 is a schematic diagram showing the gene cassette including a loxP-Venus gene as constructed above. In FIG. 6, SA denotes a splicing acceptor sequence to allow the gene cassette to accept transcription by a ROSA promoter, STOP denotes a sequence of three SV40 poly A signal cassettes linked together, Pause denotes a sequence of a human α2 globin gene RNA polymerase II transcriptional pause signal, Venus denotes a Venus sequence, pA denotes a 3′-UTR (untranslated region) of a Rabbit β-globin gene, and PuroR denotes a gene cassette composed of a SV40 promoter, puromycin N-acetyltransferase, and pA (poly A) of herpes simplex virus thymidine kinase HSV-TK).

### Reference Example 3

(Construction of Gene Cassette Including loxP-DTA Gene)

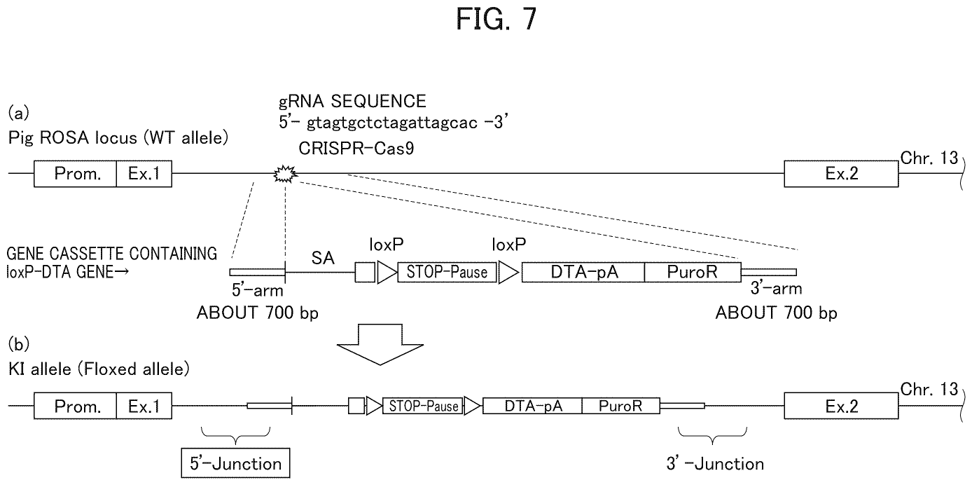

A knock-in vector composed of a splicing acceptor sequence, followed by a loxP-STOP-Pause-loxP sequence, and DTA-pA and PuroR (puromycin resistance gene cassette) for drug selection downstream thereof was constructed. Sequences of a ROSA region (about 700 base pairs each) as homology arms for knock-in are linked to both ends (5′ and 3′ sides) of the vector. FIG. 7(a) is a schematic diagram of a gene cassette including a loxP-DTA gene as constructed above. In FIG. 7(a), SA denotes a splicing acceptor sequence to facilitate transcription by a ROSA promoter, STOP denotes a sequence of three SV40 poly A signal cassettes linked together, Pause denotes a sequence of a human α2 globin gene RNA polymerase II transcriptional pause signal, DTA denotes a sequence of a diphtheria toxin A fragment, pA denotes a 3′-UTR (untranslated region) of a Rabbit β-globin gene, and PuroR denotes a gene cassette composed of a SV40 promoter, puromycin N-acetyltransferase, and pA (poly A) of herpes simplex virus thymidine kinase HSV-TK). FIG. 7(b) is a schematic diagram showing knock-in of the above-mentioned gene cassette into a ROSA region.

### Reference Example 4

(Establishment of Cells Transfected with Gene Cassette Including loxP-DTA Gene in ROSA Region by CRISPR/Cas9)

A RNP complex composed of the gene cassette including a loxP-DTA gene, a gRNA complex, and a recombinant Cas9 was introduced into 5.0×10⁵ fetal pig fibroblasts by electroporation. Here, the gRNA complex is composed of a tracrRNA (Trans-activating crRNA) and a porcine ROSA region-gRNA (guide RNA) represented by the below-mentioned sequence. A sequence that the below-mentioned porcine ROSA region-gRNA (guide RNA) has is a sequence located on porcine chromosome 13 from 65,757,140 to 65,757,159 and present between exons 1 and 2 of ROSA. However, position numbers in the above-mentioned gene are the position numbers in the UCSC Genome Browser as of May 24, 2022, and may change as the database is updated.

### Pig ROSA Region-gRNA

(SEQ ID NO: 8)
5′-GTAGTGCTCTAGATTAGCAC-3′

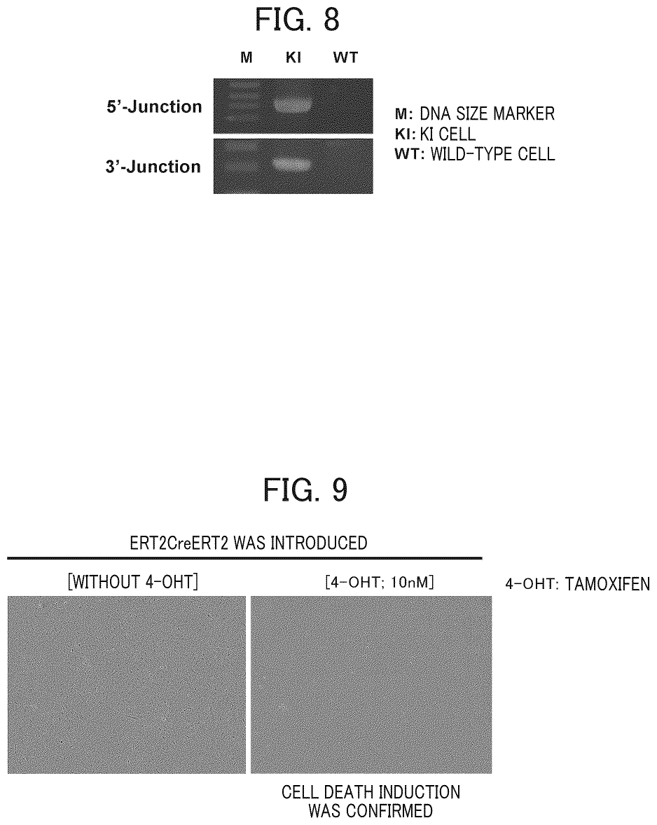

After selection for puromycin resistance, limiting dilution to less than one cell per well was performed, genetic analysis was performed on clones derived from a proliferated single cell, and genetically modified pig cells in which the gene cassette including a loxP-DTA gene as constructed above had been introduced into the porcine ROSA region were established. FIG. 8 shows electrophoretic diagrams showing confirmed results of knock-in of the above-mentioned gene cassette into a ROSA region. As is clear from the results shown in FIG. 8, the gene cassette was confirmed to be knocked into the ROSA region.

(Confirmation of Induction of Cell Death by ERT2CreERT2 and Tamoxifen in Genetically Modified Pig Cells in which Gene Cassette Including loxP-DTA Gene has been Introduced into Porcine ROSA Region)

An ERT2CreERT2 expression vector constructed separately was introduced by electroporation into the genetically-modified pig-cells in which the gene cassette including a loxP-DTA gene had been introduced into the porcine ROSA region. The resultant was then cultured in a medium supplemented with 10 nM tamoxifen (4-hydroxytamoxifen (4-OHT)). The results are shown in Table 9. As is clear from the results shown in FIG. 9, cell death of the genetically-modified pig-cells in which the gene cassette including a loxP-DTA gene had been introduced into the porcine ROSA region was confirmed.

(Construction of Genetically Modified Cloned Pig from Genetically Modified Pig Cell)

The genetically modified pig cells in which the gene cassette including a loxP-DTA gene had been introduced into the porcine ROSA region were seeded at 5×10⁵ cells in a Dulbecco's modified Eagle medium (DMEM) supplemented with 10% fetal bovine serum (FBS) on a dish having a diameter of 35 mm and passaged once. Half of the medium was removed at 14 to 18 hours after the passage, the remaining medium was cultured for 2 hours, gently pipetted, collected, and then centrifuged at 1000 rpm for 3 minutes to collect the cells. This was used as a nuclear donor cell for nuclear transplantation.

For the nuclear transplantation, denucleated unfertilized eggs in the second meiotic metaphase obtained by in vitro maturation of ova collected from an ovary of a wild-type female pig were used as a recipient cytoplasm. Nuclear transplantation manipulation was performed in an ovum manipulation solution. Electrofusion was used to fuse the nuclear donor cells with the recipient cytoplasm. Eggs that had been fused were cultured in a culture medium for 30 minutes to 1 hour and then subjected to DC electrical stimulation. At least some of the eggs were normally activated and initiated development.

Embryos normally divided on days 1 to 2 or embryos developed into blastocysts on days 5 to 6 out of the thus-in vitro cultured nuclear-transplanted embryos were transplanted to oviducts or uteri of recipient pigs with synchronized estrus cycles.

Neonatal pigs were born from at least some of the thus-transplanted embryos, and genetically modified cloned pigs could be constructed.

### Example 1

(Establishment of Cells Transfected with Gene Cassette Including loxP-Venus Gene in ROSA Region by CRISPR/Cas9)

Transgenic pig cells heterozygously transformed with the above-mentioned gene were established from the “transgenic porcine fetuses heterozygously transformed with the tissue- or organ-specific CreERT2 gene” obtained in <Reference Example 1> above. Establishment of the transgenic pig cells was performed according to a routine method. That is, skin was aseptically taken from the bodies of the fetuses and then mildly ground. The thus-ground skin tissue was filtered through a filter (300 to 500 μm aperture), suspended in a medium, and seeded into a culture flask. MEMα or DMEM including 10 to 20% (v/v) fetal bovine serum was used as the medium. After culture for 5 to 14 days, they were frozen and stored according to a routine method.

A RNP complex composed of the gene cassette including a loxP-Venus gene as constructed above in <Reference Example <2>, a gRNA complex, and a recombinant Cas9 was introduced into 5.0×10⁵ cells of the above-mentioned established transgenic pig cells by electroporation using the Neon Transfection System (Thermo Fisher Scientific). Here, the gRNA complex is composed of a tracrRNA (Trans-activating crRNA) and the porcine ROSA region-gRNA (guide RNA) represented by SEQ ID NO: 8. The sequence that the above-mentioned porcine ROSA region-gRNA (guide RNA) has is a sequence located on porcine chromosome 13 from 65,757,140 to 65,757,159 and present between exons 1 and 2 of ROSA. However, position numbers in the above-mentioned gene are the position numbers in the UCSC Genome Browser as of May 24, 2022, and may change as the database is updated. After selection for puromycin resistance, limiting dilution to less than one cell per well was performed, genetic analysis was performed on clones derived from a proliferated single cell, and genetically modified pig cells in which the gene cassette including a loxP-Venus gene as constructed above had been heterozygously introduced into the porcine ROSA region were established.

(Construction of Genetically Modified Cloned Pig from Genetically Modified Pig Cell)

The genetically modified pig cells in which the gene cassette including a loxP-Venus gene had been introduced into the porcine ROSA region were seeded at 5×10⁵ cells in a Dulbecco's modified Eagle medium (DMEM) supplemented with 10% fetal bovine serum (FBS) on a dish having a diameter of 35 mm and passaged once. Half of the medium was removed at 14 to 18 hours after the passage, the remaining medium was cultured for 2 hours, gently pipetted, collected, and then centrifuged at 1000 rpm for 3 minutes to collect the cells. This was used as a donor nucleus for nuclear transplantation.

For the nuclear transplantation, denucleated unfertilized eggs in the second meiotic metaphase obtained by in vitro maturation of ova collected from an ovary of a wild-type female pig were used as a recipient cytoplasm. Nuclear transplantation manipulation was performed in an ovum manipulation solution. Electrofusion was used to fuse nuclear donor cells with the recipient cytoplasm. Eggs that had been fused were cultured in a culture medium for 30 minutes to 1 hour and then subjected to DC electrical stimulation. At least some of the eggs were normally activated and initiated development.

Embryos normally divided on days 1 to 2 or embryos developed into blastocysts on days 5 to 6 out of the thus-in vitro cultured nuclear-transplanted embryos were transplanted to oviducts or uteri of recipient pigs with synchronized estrus cycles. Neonatal pigs were born from at least some of the thus-transplanted embryos, and it was possible to construct normally developing fetuses of genetically modified cloned pigs. Venus fluorescence can be observed in a specific tissue or organ in a conditioned manner by administering tamoxifen to female pigs carrying the above-mentioned normally developed fetuses, or by transplanting the metanephros taken from the above-mentioned normally developed fetuses into immunodeficient mice and then administering tamoxifen to the mice, or culturing the thus-taken metanephros in vitro and adding tamoxifen to the culture medium therefor.

### Example 2

(Establishment of Cells Transfected with Gene Cassette Including loxP-DTA Gene in ROSA Region by CRISPR/Cas9)

Transgenic pig cells heterozygously transformed with the above-mentioned gene were established from the “transgenic porcine fetuses heterozygously transformed with the tissue- or organ-specific CreERT2 gene” obtained in <Reference Example 1> above. Establishment of the transgenic pig cells was performed according to a routine method. That is, skin was aseptically taken from the bodies of the fetuses and then mildly ground. The thus-ground skin tissue was filtered through a filter (300 to 500 μm aperture), suspended in a medium, and seeded into a culture flask. MEMα or DMEM including 10 to 20% (v/v) fetal bovine serum was used as the medium. After culture for 5 to 14 days, they were frozen and stored according to a routine method.

A RNP complex composed of the gene cassette including a loxP-DTA gene as constructed above in <Reference Example <3>, a gRNA complex, and a recombinant Cas9 was introduced into 5.0×10⁵ cells of the above-mentioned established transgenic pig cells by electroporation using the Neon Transfection System (Thermo Fisher Scientific). Here, the gRNA complex is composed of a tracrRNA (Trans-activating crRNA) and the porcine ROSA region-gRNA (guide RNA) represented by SEQ ID NO. 8. A sequence that the below-mentioned porcine ROSA region-gRNA (guide RNA) has is a sequence located on porcine chromosome 13 from 65,757,140 to 65,757,159 and present between exons 1 and 2 of ROSA. However, position numbers in the above-mentioned gene are the position numbers in the UCSC Genome Browser as of May 24, 2022, and may change as the database is updated. After selection for puromycin resistance, limiting dilution to less than one cell per well was performed, genetic analysis was performed on clones derived from a proliferated single cell, and genetically modified pig cells in which the gene cassette including a loxP-DTA gene as constructed above had been heterozygously introduced into the porcine ROSA region were established.

(Construction of Genetically Modified Cloned Pig from Genetically Modified Pig Cell)

The genetically modified pig cells in which the gene cassette including a loxP-DTA gene had been introduced into the porcine ROSA region were seeded at 5×10⁵ cells in a Dulbecco's modified Eagle medium (DMEM) supplemented with 10% fetal bovine serum (FBS) on a dish having a diameter of 35 mm and passaged once. Half of the medium was removed at 14 to 18 hours after the passage, the remaining medium was cultured for 2 hours, gently pipetted, collected, and then centrifuged at 1000 rpm for 3 minutes to collect the cells. This was used as a donor nucleus for nuclear transplantation.

For the nuclear transplantation, denucleated unfertilized eggs in the second meiotic metaphase obtained by in vitro maturation of ova collected from an ovary of a wild-type female pig were used as a recipient cytoplasm. Nuclear transplantation manipulation was performed in an ovum manipulation solution. Electrofusion was used to fuse nuclear donor cells with the recipient cytoplasm. Eggs that had been fused were cultured in a culture medium for 30 minutes to 1 hour and then subjected to DC electrical stimulation. At least some of the eggs were normally activated and initiated development.

Embryos normally divided on days 1 to 2 or embryos developed into blastocysts on days 5 to 6 out of the thus-in vitro cultured nuclear-transplanted embryos were transplanted to oviducts or uteri of recipient pigs with synchronized estrus cycles. Neonatal pigs were born from at least some of the thus-transplanted embryos, and it was possible to construct normally developing fetuses of genetically modified cloned pigs.

### Comparative Example

(Construction of all-In-One Type Vector 1 Including Both mSix2 Promoter and CreERT2, and loxP-DTA Gene)

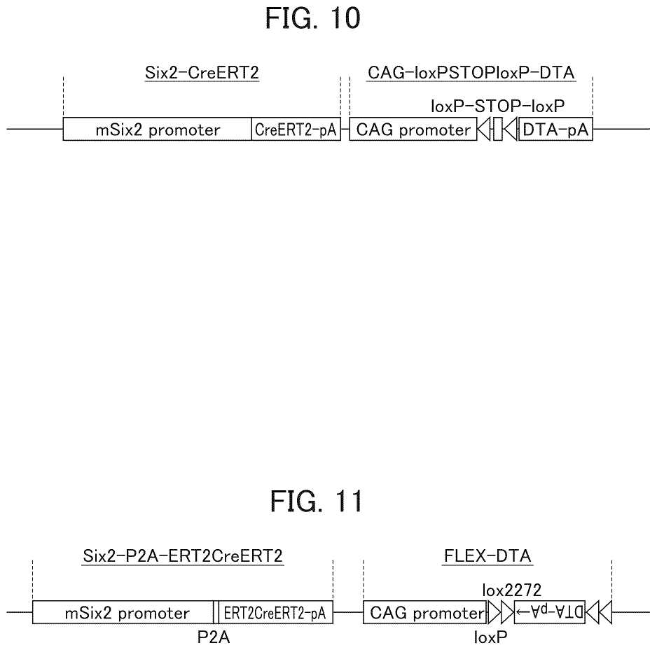

An all-in-one type vector composed of a mSix2 promoter, which is about 7 kilobases upstream of a start codon peripheral region of a mouse Six2 gene, CreERT2 and pA linked to a 3′ end of the mSix2 promoter, a CAG promotor linked downstream of the pA, a loxP-STOP-loxP sequence at a 3′ end of the CAG promotor, and DTA-pA downstream of the loxP-STOP-loxP sequence was constructed. FIG. 10 is a schematic diagram showing an all-in-one type vector 1 including both a mSix2 promoter and CreERT2, and a loxP-DTA gene as constructed above.

(Construction of all-In-One Type Vector 2 Including Both mSix2 Promoter and CreERT2, and loxP-DTA Gene)

An all-in-one type vector 2 composed of a mSix2 promoter, which is about 7 kilobases upstream of a start codon peripheral region of a mouse Six2 gene, P2A ((porcine teschovirus-1-derived self-cleaving 2A peptide) and further ERT2CreERT2 and pA linked to a 3′ end of the mSix2 promoter, a CAG promotor linked downstream of the pA, and a DTA-pA sequence (DTA-pA) (opposite orientation) sandwiched between FLEx switch-type loxPlox2272 and loxPlox2272 (opposite orientation) at a 3′ end of the CAG promoter was constructed. FIG. 11 is a schematic diagram showing an all-in-one type vector 2 including both a mSix2 promoter and CreERT2, and a loxP-DTA gene as constructed above. The resulting all-in-one type vectors 1 and 2 including both the mSix2 promoter and the CreERT2, and the loxP-DTA gene were linearized and used for ICSI as mentioned below.

(Construction of Genetically Modified Pigs with all-In-One Type Vectors 1 and 2 Via ICSI)

A transgenic pig was constructed via ICSI in the same manner as in Example 1, except that the “tissue- or organ-specific CreERT2 expression vector” was changed to the all-in-one type vector 1 or 2 including both the mSix2 promoter and CreERT2, and a loxP-DTA gene. Results of construction of the transgenic pig with all-in-one type vectors 1 and 2 via ICSI are summarized in Tables 1 and 2.

TABLE 1
Results of transgenic pig using all-in-one type vector 1 via ICSI

| | | | | | Number of | | |
|---|---|---|---|---|---|---|---|
| | Number of | | | Number of | normally | | Number of |
| Number of | transplanted | | Fetal | normally | developed | Number of | regressed |
| cultured | embryos | Recipient | age(s) | developed | transgenic | regressed | transgenic |
| embryos | (age(s) in day) | pig No. | in day | fetuses | fetuses | fetuses | fetuses |
| 1028 | 283 | #1825, | 28 to 36 | 20 | 1 | 17 | 3 |
| | (5 or 6 | #1826, | days old | | | | |
| | days old) | #1852, | | | | | |
| | | #1903 | | | | | |

As is clear from Table 1, ICSI-mediated gene transfer with the all-in-one type vector 1 resulted in one normally developed transgenic fetus, but a production efficiency of transgenic pig fetuses was extremely low at less than 0.35%. Imbalance (excess) of a copy number of the loxP-DTA gene relative to a copy number of the CreERT2 gene may have been a factor.

TABLE 2
Results of construction of transgenic pig using all-in-one type vector 2 via ICSI

| | | | | | Number of | | |
|---|---|---|---|---|---|---|---|
| | Number of | | | Number of | normally | | Number of |
| Number of | transplanted | | Fetal | normally | developed | Number of | regressed |
| cultured | embryos | Recipient | age(s) | developed | transgenic | regressed | transgenic |
| embryos | (age(s) in day) | pig No. | in day | fetuses | fetuses | fetuses | fetuses |
| 776 | 343 | #1850, | 35 or 36 | 22 | 0 | 38 | 5 |
| | (2, 5, or 6 | #1851, | days old | | | | |
| | days old) | #1904 | | | | | |

As is clear from Table 2, ICSI-mediated gene transfer with the all-in-one type vector 2 produced no normally developed transgenic fetuses (production efficiency of transgenic pig fetuses was 0%). In addition, all transgenic fetuses regressed.

As mentioned above, it was difficult to construct a transgenic pig with the all-in-one type vector including both the mSix2 promoter and the CreERT2, and the loxP-DTA gene.

1. A method for producing a genetically modified pig, the method comprising:
introducing a gene cassette comprising a construct into a safe harbor region in a first transgenic cell, the construct comprising a gene encoding a functional protein, the functional protein being expressible by an action of a recombinant enzyme,
the first transgenic cell comprising a promoter expressed in a specific tissue or organ, and a gene encoding the recombinant enzyme, the gene being downstream of and under control of the promoter expressed in a specific tissue or organ.

2. The method according to claim 1, wherein the safe harbor region is a ROSA region.

3. The method according to claim 1, wherein the vector is a plasmid vector or an artificial chromosome vector.

4. The method according to claim 1, comprising establishing a second transgenic pig cell by introducing the gene cassette into the safe harbor region.

5. The method according to claim 1, comprising subjecting an established second transgenic pig cell to somatic cell cloning to obtain the genetically modified pig.

6. A somatic cell established from a genetically modified pig produced by the method according to claim 1.

7. (canceled)

8. The method according to claim 1, wherein the first transgenic cell is a transgenic cell established from a transgenic pig having been transformed by way of microfertilization of an egg with a solution comprising a vector and pig sperm, and
the vector comprises the promoter expressed in the specific tissue or organ, and the gene encoding the recombinant enzyme, the gene being downstream of and under control of the promoter expressed in the specific tissue or organ.


SEQUENCE LISTING (16 sequences)

SEQ ID NO: 1 - DNA, 34 bp, synthetic construct
  misc_feature 1..34: note = loxP
  source 1..34: mol_type = other DNA
ataacttcgtatagcatacattatacgaagttat

SEQ ID NO: 2 - DNA, 34 bp, synthetic construct
  misc_feature 1..34: note = lox511
  source 1..34: mol_type = other DNA
ataacttcgtatagtatacattatacgaagttat

SEQ ID NO: 3 - DNA, 34 bp, synthetic construct
  misc_feature 1..34: note = lox2272
  source 1..34: mol_type = other DNA
ataacttcgtataggatactttatacgaagttat

SEQ ID NO: 4 - DNA, 34 bp, synthetic construct
  misc_feature 1..34: note = loxFAS
  source 1..34: mol_type = other DNA
ataacttcgtatatacctttctatacgaagttat

SEQ ID NO: 5 - DNA, 34 bp, synthetic construct
  misc_feature 1..34: note = lox RE
  source 1..34: mol_type = other DNA
ataacttcgtatagcatacattatacgaacggta

SEQ ID NO: 6 - DNA, 34 bp, synthetic construct
  misc_feature 1..34: note = lox LE
  source 1..34: mol_type = other DNA
taccgttcgtatagcatacattatacgaagttat

SEQ ID NO: 7 - DNA, 34 bp, synthetic construct
  misc_feature 1..34: note = FRT
  source 1..34: mol_type = other DNA
gaagttcctattctctagaaagtataggaacttc

SEQ ID NO: 8 - DNA, 20 bp, synthetic construct
  misc_feature 1..20: note = ROSA-gRNA
  source 1..20: mol_type = other DNA
gtagtgctctagattagcac

SEQ ID NO: 9 - DNA, 23 bp, synthetic construct
  misc_feature 1..23: note = CreERT2_1stF
  source 1..23: mol_type = other DNA
catgtccatgctgcccaccttcg

SEQ ID NO: 10 - DNA, 21 bp, synthetic construct
  misc_feature 1..21: note = CreERT2_1stR
  source 1..21: mol_type = other DNA
gtatatcctggcagcgatcgc

SEQ ID NO: 11 - DNA, 20 bp, synthetic construct
  misc_feature 1..20: note = CreERT2_2ndF
  source 1..20: mol_type = other DNA
tcgcaagaacctgatggaca

SEQ ID NO: 12 - DNA, 20 bp, synthetic construct
  misc_feature 1..20: note = CreERT2_2ndR
  source 1..20: mol_type = other DNA
cgccgcataaccagtgaaac

SEQ ID NO: 13 - DNA, 21 bp, synthetic construct
  misc_feature 1..21: note = PigSix2_1stF
  source 1..21: mol_type = other DNA
agtcgcagcgtgctgcgcgag

SEQ ID NO: 14 - DNA, 20 bp, synthetic construct
  misc_feature 1..20: note = PigSix2_1stR
  source 1..20: mol_type = other DNA
ctaggagcccaggtccacga

SEQ ID NO: 15 - DNA, 21 bp, synthetic construct
  misc_feature 1..21: note = PigSix2_2ndF
  source 1..21: mol_type = other DNA
accacgcaggtcagcaactgg

SEQ ID NO: 16 - DNA, 21 bp, synthetic construct
  misc_feature 1..21: note = PigSix2_2ndR
  source 1..21: mol_type = other DNA
tccgagctgcctagcaccgac